(19) United States
(12) Patent Application Publication
(10) Pub. No.: US 2024/0156660 A1
(43) Pub. Date: May 16, 2024

(54) MEDICAL CART FOR MAGNETIC TREATMENT OF MIDDLE EAR CONDITIONS

(71) Applicant: Otomagnetics, Inc., Gaithersburg, MD (US)

(72) Inventor: Benjamin Shapiro, Washington, DC (US)

(21) Appl. No.: 18/334,975

(22) Filed: Jun. 14, 2023

Related U.S. Application Data
(63) Continuation of application No. PCT/US2021/072938, filed on Dec. 15, 2021.
(60) Provisional application No. 63/125,825, filed on Dec. 15, 2020.

(51) Int. Cl. A61G 12/00; A61G 13/12; A61K 41/00; A61M 31/00; A61N 2/06

(52) U.S. Cl. CPC A61G 12/001; A61G 13/121; A61K 41/0052; A61M 31/00; A61N 2/06; A61M 2210/0668

(57) ABSTRACT

A medical cart for administering a magnetic agent to a patient through the ear canal is disclosed. The medical cart comprises a magnet assembly array that is configured to direct a magnetic agent into the middle ear space of the patient's ear. The magnet assembly is further configured to produce magnetic force that directs the magnetic agent to a second administration ear and opposes the force of gravity to hold the magnetic agent in the first administration ear.

### CROSS REFERENCE TO RELATED APPLICATIONS

The present application claims priority under 35 U.S.C. § 119(e) to U.S. Provisional Patent Application No. 63/125,825, titled MEDICAL CART FOR MAGNETIC TREATMENT filed on Dec. 15, 2020, the disclosure of which is herein incorporated by reference in its entirety.

### BACKGROUND

The present disclose as directed to a medical treatment cart or beds for administering magnetic therapies. In order to magnetically deliver therapy (e.g. drugs, proteins, or genes) to physical targets in a patient's body, the magnetic force on the magnetic nanoparticles carrying the therapy must be positioned in a specific direction. In particular, the magnetic force must deliver the magnetic nanoparticles from where they are first placed (e.g. by a dropper, syringe, pipette, via a gel or swab, or by other methods) to the desired target. For example, in the context of treating middle ear infections in patients, the magnetic nanoparticles can be placed by the clinician in the outer ear (the ear canal) by a dropper, and then the magnetic force pushes and transports the particles from the outer ear, through the ear drum (tympanic membrane), and into the middle ear space. The magnetic force is configured so that it is directionally pointing from the middle ear. In order to produce a magnetic force in these specific directions, the magnets must be configured in such a way that the resulting orientation of the magnetic force matches both the anatomy of a patient and the direction from an administration location to region therapy target location.

Further, the magnetic field must be strong enough to transfer the magnetic nanoparticles with their therapeutic payload from an administration location, through intervening tissue, to the desired target location. For example, the administration location may be the outer ear, where the agent is directed through the ear drum, and reaches the desired target location such as the middle ear space. In the present example, the intervening tissue is a strong barrier (similar to skin, the ear drum is a protective barrier in the human body) and requires a strong magnetic force to pass through the tissue barrier. Additionally, the distance from magnets to nanoparticles may be significant (for a magnetic to produce sufficient pull force to align correctly from outer to middle ear, the magnets must be placed behind the head), and therefore the magnet may be substantial in size to produce this type of force (e.g. at least the width of the head). The strong magnetic force may require a strong magnet, and larger magnets or a large assembly of multiple magnets may provide such a strong force. However, large magnets are heavy, and it may therefore be desirable to provide for a device or system that can safely and conveniently accommodate a strong and heavy magnet or assembly of magnets. Embedding the magnet or magnet assembly in a cart is a possible solution, as disclosed herein.

Further, the orientation of the patient and the direction of the magnetic force on the magnetic particles may need to respect other constraints, both based on anatomy and physics. In particular, in die context of treating middle ear infections, the magnetic nanoparticles may be placed in liquid solution to form a liquid formulation. That formulation may be administered to the outer ear vis a dropper as a liquid. During the magnetic transfer treatment, the liquid solution must remain in the outer ear. Thus the patient's head should be so orientated that the force of gravity does not cause the liquid formulation of magnetic nanoparticles to leak out from the outer ear canal during the administration of the magnetic force. Moreover, it may be desired that the magnetic particles are pressed up against the ear drum (tympanic membrane) by gravity (plugging the ear with was would not achieve such a purpose). Such needs provide constraints on how the patient may be oriented, in particular the patient's head should be oriented so that the ear canal does not point down (in which case liquid would drain out of the ear canal).

In addition, patient comfort is important, as is ease of use for the doctor or clinician administering the therapy. This also places constraints and restrictions on how the patient may be arranged, how the clinician approaches them, and that in turn has implications for system design. In particular, disclosed herein is a medical cast configured to be comforting to a patient, easy to use by the clinician, and meets the needs of delivering the therapy magnetically.

In addition, when treating middle ear infections, it is common to have to treat both ears. It is desirable to be able to treat both ears in one session. Suppose the patient's left ear is treated first. That means the cart disclosed below has applied a magnetic force on magnetic particles placed in the left ear canal, to transfer them through the left ear drum (left tympanic membrane), and into the left middle ear space. Next the right ear is treated The magnetic force that is applied during treatment of the right car, that magnetic force should not act to pull magnetic particles back out of the left middle ear (i.e. it should not act to even partially reverse the treatment of the left ear that has just been concluded). This imposes yet another constraint on the direction of magnetic forces that can be applied to the magnetic particles as the patient is being treated.

### SUMMARY

The present invention provides for systems and methods to magnetically deliver therapy to middle ear spaces in patients The disclosed cart system enables a patient's head alignment, and a matching alignment of magnetic forces, such that these forces can transport magnetic nanoparticles with attached therapy (drugs, proteins, or genes) from the outer ear, through the ear drum into middle ear spaces The magnet assembly encased in the cart is large enough to provide sufficient magnetic force. Further, the system allows treatment of both ears, with treatment of the second ear not disrupting preceding treatment of the first ear The systems provides for patient comfort, for effective and easy use of the system by a doctor or clinician.

In one aspect of the present disclosure, a magnetic agent delivery cart comprising: an upper bed platform, a magnetic assembly located under the upper bed platform, wherein the magnet assembly is centered underneath the longitudinal axis of the upper bed platform and aligned at a proximal end of the magnetic agent delivery cart: one or more side fairings configured to hide the magnetic assembly, wherein the one or more side fairings is removable; a headrest located on top of the upper bed platform and positioned above the center of the magnet assembly, the headrest further comprising: a first head contour and a second head contour, wherein the first head contour is configured for die patient in a first administration position and the second bead contour is configured for the patient in a second administration position; and an alignment grid configured according to the first and second administration position and the location of the magnet assembly, wherein the alignment grid comprises a first position vertical line that corresponds to the sagittal plane of the patient in the first administration position, a second position vertical line that corresponds to the sagittal plane of the patient in the second administration position, and a horizontal line that corresponds to the transverse plane along the patient's eyes.

In another aspect of the present disclosure, a system for delivering a magnetic agent to a patient through the ear canal, the system comprising: a magnetic therapeutic agent comprising a magnetic nanoparticles attached to a therapy: a medical cart comprising an upper bed platform, a magnetic assembly located under the upper bed platform, wherein the magnet assembly is centered underneath the longitudinal axis of the upper bed platform and aligned at a proximal end of the magnetic agent delivery cart; one or more side fairings configured to hide the magnetic assembly, wherein the one or more side fairings is removable; a headrest located on top of the upper bed platforms and positioned above the center of the magnet assembly, the headrest further comprising: a first head contour and a second head contour, wherein the first bead contour is configured for the patient in a first administration position and the second head contour is configured for the patient in a second administration position; and an alignment grid configured according to the first and second administration position and the location of the magnet assembly, wherein the alignment grid comprises a first position vertical line that corresponds to the sagittal plane of the patient in the first administration position, a second position vertical line that corresponds to the sagittal plane of the patient in the second administration position, and a horizontal line that corresponds to the transverse plane along the patient's eyes.

In another aspect of the present disclosure, a method for delivering a magnetic agent to a patient through the ear canal, the method comprising, positioning a patient on a medical cart in a first administration position, wherein the medical cart comprises a magnet assembly and a headrest, and wherein the headrest comprises a first head contour and a second bead contour, and wherein the headrest comprises a horizontal alignment line, first position vertical alignment line, and a second position vertical alignment line; aligning the bridge of the patient's nose with the vertical first position indicator; aligning the patient's eyes in the horizontal plane with a horizontal position indicator, rotating the patient's head so that a first administration ear is above the horizontal plane; administering a magnetic therapeutic agent to the first administration ear; pushing, by the magnet assembly, a magnetic therapeutic agent to a middle ear space of the patient's first car; repositioning the patient on the medical cart in a second administration position; aligning the bridge of the patient's nose with the vertical second position indicator; aligning the patient's eyes in the horizontal plane with a horizontal position indicator; rotating the patient's head so that a second administration ear is above the horizontal plane; administering the magnetic therapeutic agent to the second administration ear; pushing, by the magnet assembly, the magnetic therapeutic agent to a middle ear space of the patient's second ear; and pushing, by the magnet assembly, the magnetic therapeutic agent in the first administration ear so that is remains in the middle ear space of the patient's second ear.

In yet another aspect of the present disclosure, a method for delivering a magnetic agent to a patient through the ear canal, the method comprising: determining, based on the diameter of a patient's head, a headrest that conforms the patient's head, wherein the headrest comprises a first head contour and a second head contour, and wherein the headrest comprises a horizontal alignment line, first position vertical alignment line, and a second position vertical alignment line; aligning the headrest on the medical cart based on one or more alignment marker that is relative to the magnet assembly; positioning the patient on the medical cart in a first administration position, wherein the medical cart comprises a magnet assembly and the headrest; aligning the bridge of the patient's nose with the vertical first position indicator; aligning the patient's eyes in the horizontal plane with a horizontal position indicator; rotating the patient's head so that a first administration ear is above the horizontal plane; administering a magnetic therapeutic agent to the first administration ear; pushing, by the magnet assembly, a magnetic therapeutic agent to a middle ear space of the patient's first ear; repositioning the patient on the medical cart in a second administration position; aligning the bridge of the patient's nose with the vertical second position indicator; aligning the patient's eyes in the horizontal plane with a horizontal position indicator; rotating the patient's head so that a second administration ear is above the horizontal plane; administering the magnetic therapeutic agent to the second administration ear; pushing, by the magnet assembly, the magnetic therapeutic agent to a middle ear space of the patient's second ear; and pushing, by the magnet assembly, the magnetic therapeutic agent in the first administration ear so that is remains in the middle ear space of the patient's second ear.

### BRIEF DESCRIPTION OF THE DRAWINGS

FIG. 1 shows a cross-sectional view of a patient's head and the anatomy of the left ear and right ear, in accordance with at least one aspect of the present disclosure.

FIG. 2 shows a cross-sectional view of a patient in a first and second agent delivery position on a medical cart, in accordance with at least one aspect of the present disclosure.

FIG. 3 shows a system for administering an agent to a pediatric patient comprising a medical cart, in accordance with at least one aspect of the present disclosure.

FIGS. 4A and 4B illustrate a three dimension view and profile view of a magnetic assembly comprising a magnet of agnetic array, in accordance with at least one aspect of the present disclosure.

FIGS. 5A-5D shows a transparent alignment grid at the surface of the headrest, in accordance with at least one aspect of the present disclosure.

FIG. 6 shows a top view of the medical cart comprising safety bars or guard rails, in accordance with at least one aspect of the present disclosure.

FIG. 7 shows a profile view of the medical cart comprising a cantilever leg that may be folding up or down, in accordance with at least one aspect of the present disclosure.

FIGS. 8A-8N illustrates a treatment procedure for administering a magnetic therapeutic agent to a patient through the ear canal, in accordance with at least one aspect of the present disclosure.

FIG. 9 shows a flow diagram for administering a magnetic therapeutic agent to a patient on a medical cart, in accordance with at least one aspect of the present disclosure.

FIG. 10 shows a flow diagram for configuring a medical cast and administering a magnetic therapeutic agent to a patient on the medical cart, in accordance with at least one aspect of the present disclosure.

### DETAILED DESCRIPTION

The present disclosure describes a medical cart for treating medical conditions affecting the middle ears in patients, by using magnetic force to push the magnetic agent to a target position. In various aspects, a patient (e.g., a child or an adult) may lie or sit on the medical cart or bed and undergo a procedure of medical treatment with a magnetic agent. The magnetic agent comprises magnetic particles with attached therapies, such as drugs, proteins, or genes, and the magnetic agent is directed to the patient's middle ear or ears with magnetic forces, as disclosed in U.S. Patent Application Publication No. 2020/0146995, which is incorporated herein by reference in its entirety. In one aspect, the medical cart comprises a bed platforms, a bead rest, one or more guild rails, an alignment grid to guide alignment of the patient's head by a doctor or clinician, and other components to keep the patient comfortable (e.g. covering, cushions, etc). The medical cart further comprises a magnet assembly located beneath the bed platform, and is configured to pull magnetic particles into the middle ears of the patient. The magnetic array may be hidden by removable side coverings or fairings to provide a less intimidating appearance to the patient.

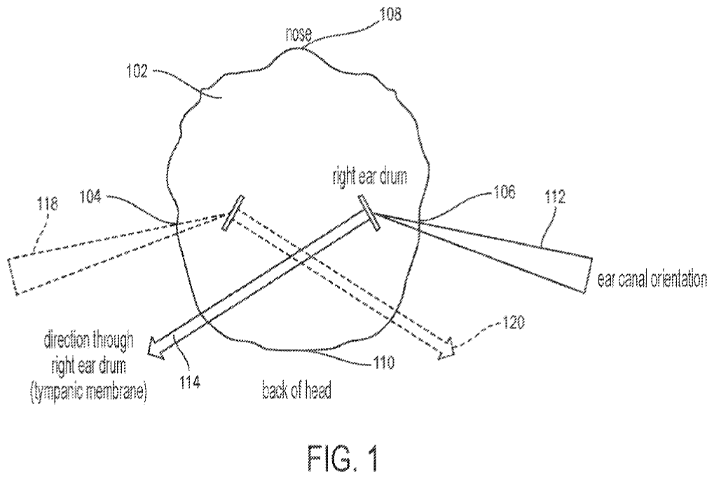

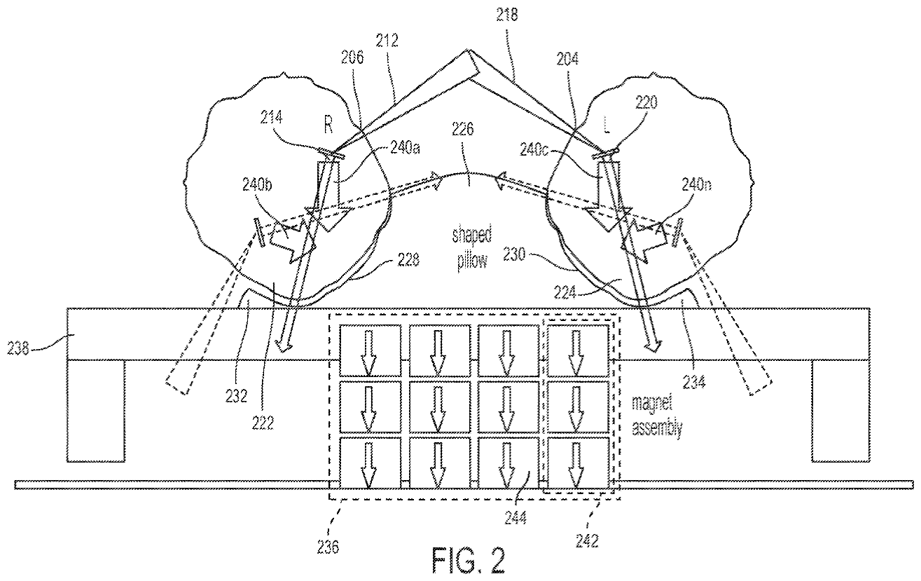

In various aspects, the medical cart is configured to treat one or both ears of the patient, and is optimized for the patient's conform during the treatment procedure. Additionally, the medical cart is designed for effective and easy use by the doctor of clinician, and can fit into a clinical setting (is safe and convenient to use, can be stowed away when not in use, etc). In one aspect, the headrest comprises an alignment grid that guides a clinician to position the patent on the medical can and aids in the administration of a magnetic therapeutic agent by the doctor or clinician. The shape of the headrest and the location of the alignment grids may be configured according to the location and proximity to the magnetic assembly, and in consideration of principles of physics (e.g. gravitational or magnetic force) and human anatomy constraints of the left and right ear, as shown in FIGS. 1 and 2.

FIG. 1 shows a cross-sectional view of a patient's head 102 and the anatomy of the left ear 104 and right ear 106. The cross-sectional view is a downward looking view from above the patient's head (transverse plane), with the nose 108 oriented in a top position and the back of the head 110 oriented in a bottom position. The orientation angle of the right ear canal 112 (outer ear) is shown by a conical marking. The orientation of the right ear drum 114 (right tympanic membrane) is marked by the arrow “direction through right ear drum”. The left ear canal orientation 118 and the left ear drum 120 are depicted with a dotted line FIG. 1 illustrates an approximate or representative anatomical orientation of a patient, and it is understood that the exact orientations may vary from patient to patient.

These anatomical characteristics, illustrated by FIG. 1, are considered for determining the patient's optimal positioning for the administration of a therapeutic agent in the left and right ear During the treatment procedure, the magnetic therapeutic agent is administered into the ear canal, through the outer ear, and is directed to the middle ear through a combination of gravitational force and magnetic force. The magnetic therapeutic agent comprises a plurality of therapeutic magnetic particles, and may be may be administered through the outer car by a dropper or syringe, in a liquid composition or formulation.

FIG. 2 illustrates a cross-sectional view of a patient in a first and second agent delivery position on a medical cart. The first position 222 is associated with administering the therapeutic agent in the patient's right ear and the second position 224 is associated with administering the therapeutic agent in the patient's left ear. The first position 222 and the second position 224 are optimal for a liquid formulation of the therapeutic agent to stay in the enter ear and not leak out due to gravity. The administration positions 222, 224 orient the respective outer ear canals in a position above die horizontal plane, such that the ear canals are not pointing at a downward angle. The headrest 226 shape comprises a first bead contour 228 to support the patient's bead in the first position 222 so that the patient can comfortably remain in the first position 222 while the magnetic and gravitation forces act upon the magnetic therapeutic agent and direct the agent to the middle ear. Similarly, the headrest 226 shape comprises a second head contour 230 for the patient to rest their head in the second position 224. The profile view of the headrest 226 resembles a normal distribution curve with wedges 232, 234 at the edges (e.g. first percentile and ninety-ninth percentile of a normal distribution) to secure the patient's head in place in the first position 222 and the second position 224. The headrest 226 may consist of a dense supportive material such as a foam or very firm memory foam.

FIG. 2 further shows a magnet assembly 236, located underneath the headrest 226, that produces a magnetic field and acts upon the magnetic therapeutic agent. In various aspects, the magnet assembly 236 may be covered by a removable fairing that prevents the magnet assembly 236 from being visible to the patient. Although middle ear conditions may affect any age group, middle ear infections are particularly prevalent in younger children. For a young child, it is desirable to have the treatment system look familiar, such as a simple bed, without showing any other features to the patient that may be intimidating to the parent or child. The removable fa rings are configured to hide the magnetic assembly and make the medical cart appear to look like a traditional bed. This can help reduce patient stress and put the patient at case. When the patient is calm, there is reduced motion and reduced likelihood that the patient's ear will drop below the horizontal plane. This ultimately allows the patient to remain in the optimal delivery position for a longer period of time and prevents the therapeutic agent from inadvertently dripping out of the administered car. Thus, as shown in FIG. 2, the magnet array 236 is located underneath the bed platform 238 and positioned under the headrest 226, such that the magnetic array 236 is not visible to the patient.

FIG. 2 further illustrates a directional magnetic force produced by the magnetic array 236. The magnetic forces 240a-n are shown by arrows which also indicate the direction of the magnetic force. The location, size, shape, and magnetization of the magnet assembly 236 are selected so that the magnetic forces 240a. 240c points substantially through the patient's ear drum (tympanic membrane) 214, 220. For example the magnetic force 240a points through the right ear drum 214, while the right ear canal orientation 212 remains above the horizontal plane.

In various aspects, both right and left ears may be treated in a single administration session. In order to treat both ears in a single session, it is necessary that once a first ear is treated, that treating the second ear does not reverse or partially reverse the treatment just completed for the first ear. For example, a patient's right ear 206 is treated first in the first treatment position 222, allowing the magnetic therapeutic agent to be pulled to the patient's right middle ear space. The patient's left ear may then be subsequently treated after the right ear. While treating the left ear 204 in the second treatment position 224, it is necessary that “stray” magnetic force does not pull the magnetic particles out of the right ear 206. The first 222 and second treatment positions 224 are specifically designed so that magnetic forces 240b and 240n keep the magnetic therapeutic agent in the previously treated ear. For example, when the patient is in the second treatment positon 224, the magnetic force 240n on the right ear 206′ is oriented substantially through the right ear drum, but the magnetic force 240c on the left ear 204 as also still oriented through the left ear drum and is not oriented in such a way as to pull particles back out of the left middle ear space.

It is further desirable that the medical cart is configured to naturally support the patient's head, so that the head is oriented according to the magnetic force produced by the magnetic assembly. Thus, the system discloses a predetermined headrest shapes that specifically positon the patient's head according gravitational and magnetic forces, for the first and second ear administration. Further, a transparent alignment grid may be visible or engraved into the headrest to aid the clinician in quickly and accurately placing the patient's head in the administration position.

In various aspects, the shape of the headrest, the location of the first and second headrest contours, and the location of alignment grid are determined according to the relative position to the magnetic assembly and the forces produced by the magnetic assembly. Accordingly, the magnetic assembly is configured according to the location, size, shape, and magnetization of magnets, to produce a desired magnetic force (e.g. 1 tesla of force experienced at the ear canal of the administration ear) on the therapeutic agent in patient's ear canals. The magnetic force, which is not the same nor equal to the magnetic field, as configured to produce directional force (vectors) for the left and right ear, in the first administration position 222 and the second administration positon 224.

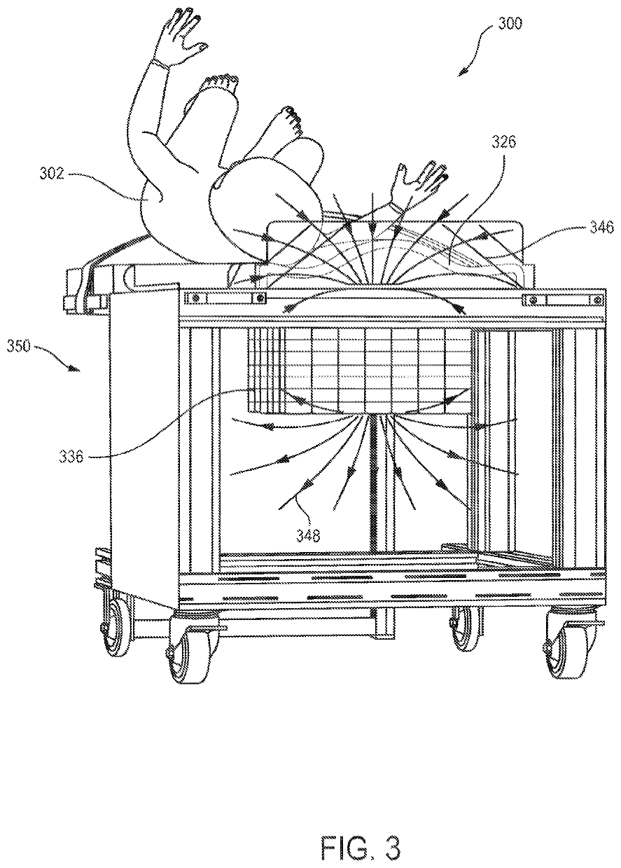

If the vector magnetic field is denoted by B, where B=[Bₓ(x, y, z), B_{y}(x, y, z), B_{z}(x, y, z)] is a 3-dimensional vector that varies across space X=(x, y, z), then the force on magnetic nanoparticles is given by F=k[dB/dx]B and is itself also a 3-dimensional vector that varies across space, i.e. F=[Fₓ(x,y,z), F_{y}(x, y, z), F_{z}(x, y, z)]. Here, dB/dX is the Jacobian matrix, and k is a coefficient whose properties depend based on the size and material parameters of the magnetic 1 nanoparticles. Alternatively and equivalently, the magnetic force F can also be written as F=1/2grad(∥B∥²) where grad is the gradient, ∥B∥ is the norm of B. We disclose that the selection of the magnet assembly was chosen so that the magnetic force F=[Fₓ(x, y, z), F_{y}(x, y, z), F_{z}(x, y, z)] points in the desired directions, meaning it matches the directions shown in FIG. 3. This selection was done by conducting a mathematical optimization over magnet element placement, to find possible magnet placements that create forces in the right directions, for intended patient bead orientations, for both ears.

FIG. 3 shows a system 300 for administering a magnetic therapeutic agent to a pediatric patient 302 comprising a medical cart 350. In the present aspect, the magnetic therapeutic agent composition comprises a magnetic nanoparticle particle, such as iron, attached to therapeutic component that can be administered to the patient. The system is configured to pull these agents into the patient's middle ears by the magnetic force that is a result of the magnetic field 348 produced by the magnetic assembly 336. In one aspect, the system 300 can be configured for patients 302 as young as 6 months old. The headrest 326 for the patient's head may be selected or configured based on the patient's head diameter. The magnet can be seen under the headrest, inside the system and may not be visible to the patient or others around the patient. The structure of the system or cart can be compact and can be on wheels, which can allow for easier storage when the system is not in use.

In one aspect, the headrest can be shaped as shown, and the magnet is under it inside the cart. The patient's natural bead position may be set by the headrest, and can orient the ears so that the magnetic force points from ear canal through the ear drum into the middle ear. The medical cart is configured to treat one ear at a time, without “untreating” the previously treated ear with the magnet assembly.

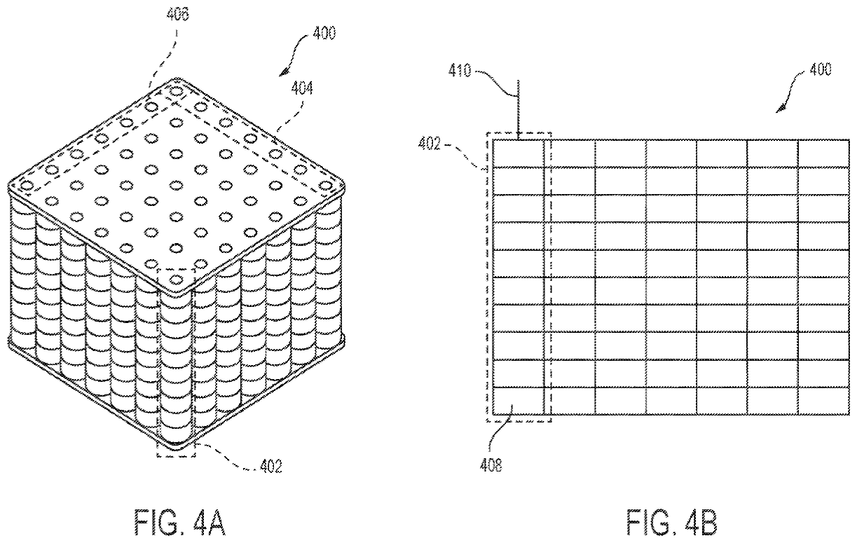

FIGS. 4A-4B illustrate a three-dimension view and profile-view of a magnetic assembly 400 comprising a magnetic array. The magnetic assembly 400 is configured to provide a predetermined magnetic force on the magnetic nanoparticles so that the therapeutic agent is directed across the ear drum. Additionally, that patient is positioned such that the direction of the magnetic forces are oriented in the same direction as both ear drums, which is different and distinct from the direction of the magnetic field. Accordingly, the magnetic field produced by the magnetic assembly must be designed for both head orientations; the first position 222 and second position 224 as illustrated m FIG. 2. Finally, the magnetic assembly may be designed and configured based on cost, case, and safety of manufacturing.

In various aspects, the magnetic 400 may comprise elect electromagnets, permanent magnets, or an array of permanent magnets. FIGS. 4A-4B illustrate and aspect of the magnetic assembly comprising of multiple strong but smaller permanent magnets, rather than a single larger and stronger magnet. Smaller magnets can be sourced more easily than one large one, and can be handled more safely during the manufacturing process. The magnetic assembly 400 of FIGS. 4A-4B comprises a magnetic array of a plurality of pillars 402 that are arranged in according to 7 rows 404 and 7 columns 406. Additionally, the magnetic army may also be configured in an analogous N rows×N columns structure, such that the N×N structure provides a predetermined amount of magnetic force on the magnetic nanoparticles to sufficient to of the magnetic agent to across the ear drum. In one aspect, the magnet assembly is configured to produce 1 tesla of force on a magnetic therapeutic agent in the ear canal of an administration ear.

In one aspect, the pillars 402 of the magnet assembly 400 may comprises a plurality of small disc magnets 408, such as Neodymium N42 disc magnets where each disc is 2 inches in diameter by 1 inch in height, with a ¼ inch hole in the middle for a supporting rod to go through. The plurality of disc magnets may be bonded together with a supporting rod 410, where each of the plurality of discs may be magnetized in the same vertical direction with opposite polarity ends attracted to each other. In one aspect, there may be 10 magnetic discs 408 for each pillar 402 in the array. In one aspect, the magnet assembly may be approximately 14 inches wide, +/−1 inch, 14 inches deep +/−1 inch, and 10 inches tall +/−1 inch.

The magnetic fields of the plurality of discs combine to create a resulting magnetic field for each pillar. Similarly, the magnetic fields of the plurality of pillars combine to create a resulting magnetic field for the magnetic assembly. Therefore, the same resulting magnetic fields may be produced by different magnetic assembly pillar and disc combinations. As discussed above, the resulting magnetic field is determined according to a calculated magnetic force on the magnetic nanoparticles.

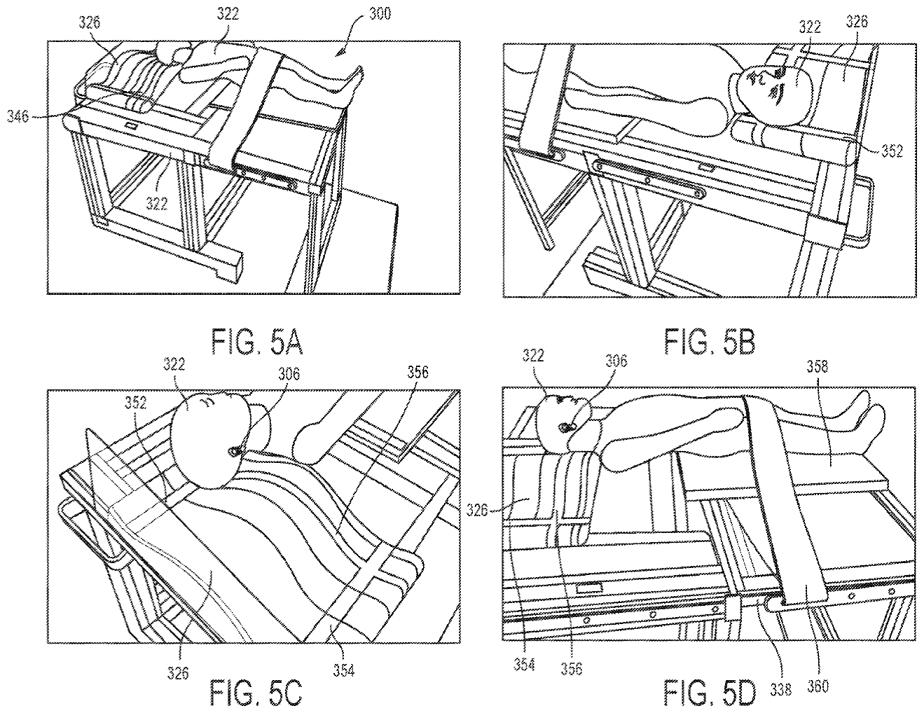

FIGS. 5A-D show a medical simulation mannequin positioned according to a transparent alignment grid 346 on the surface of a headrest 325. The alignment grid 346 allows the clinician to easily align the patient's head according to the magnetic forces, invisible to the naked eye, produced by the magnetic assembly. The alignment grid may be a light color indicator in contrast with a dark color of the headrest so that the alignment grid is shown in high contrast and can be seen through a protective covering. A protective or sanitary covering may be used in conjunction with the alignment grid and allows the clinician to view the alignment grid through the protective covering, without the patient making direct contact with the headrest. In another aspect, the alignment grid 346 may be engraved into the surface of the headrest 326. Similarly, the physical engraving may be perceived through a protective covering.

The headrest 326 may also be removable and would require one or more headrest markings to affix or align the headrest so that the alignment grid correctly aligns with the magnet assembly beneath the upper bed platform 338. It is anticipated that the clinician would stand behind or to the side of the patient and help align the patient's head with the alignment grid 346 of the headrest 326. Additionally, the headrests 326 may be different sizes for different patient head diameters and may require alignment grids to be uniquely calibrated for the shape of the headrest. In one aspect, the shape and size of the first and second head contours of the headrest are different sizes to meeting smaller or larger patients. Accordingly, the alignment grid is calibrated so that the patient's head is in the desired position for the magnetic force to act upon the magnetic therapeutic agent.

FIG. 5C shows the medical simulation mannequin in the first administration position 322 with the right ear 306 positioned for treatment. The alignment grid 346 further comprises a vertical first position indicator 352, vertical second position indicator 354, and a horizontal position indicator 356. In one aspect, the clinician aligns the patient's eyes according to the horizontal position indicator 356 and the bridge of their nose according to the vertical first position indicator 352. Additionally, die clinician aligns the patient's head so that the outer right ear 306 is above horizontal.

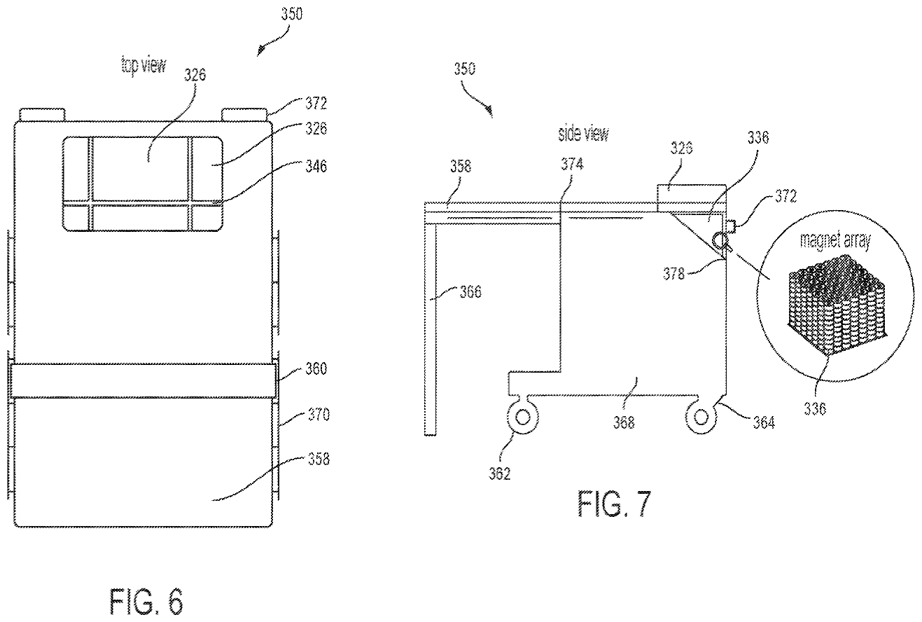

FIG. 5D shows the medical cart optionally comprising a mattress 358 positioned on top of the upper bed platform 338, an adjustable security belt 360, and guard rails 370 to help keep the patient in place. FIG. 6 further shows a top view of the medical cart 350 comprising safety bars or guard rails 370. While the patient may be secured by the adjustable security belt 360, it is desirable to further support the sides of the medical cart 350 with guard rails 370. The medical cart is configured to patient to be moved from a first position to a second position and the guard mails may prevent the patient from falling of the cart during a position transition.

FIG. 7 shows a profile view of the medical cart 350 comprising a cantilever ley 366 that may be folding op or down. In one aspect, the medical cart 350 may be a compact design so that it may fit into an existing exam room without requiring dedicated storage space. The medical cart 350 may comprises a folding hinge 374 on the upper bed platforms 338 to fold to a smaller storage size. Additionally, the medical cart 350 may further comprise one or more casters 362 for positioning and relocation of the medical cart 350. At least one of the casters 362 may be lockable with respect to transverse motion along the ground to allow the medical cant 380 to pivot about a pivoting axis perpendicular to the ground. When position is selected, that medical cart 350 is secured in place by the cantilever leg 366 and one or more caster brakes 364. Thus after use, the cart can be folded up and rolled into a storage area. FIG. 7 further shows the removable side fairings 368 covering the magnet assembly 336 from the field of view from the patient. The magnet assembly 336 is located at the proximal end 378 of the cart and is aligned in the center of the longitudinal axis of the cart.

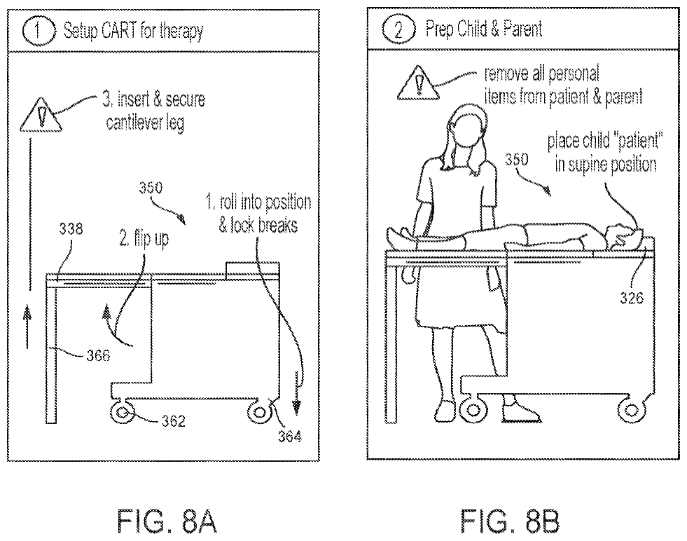

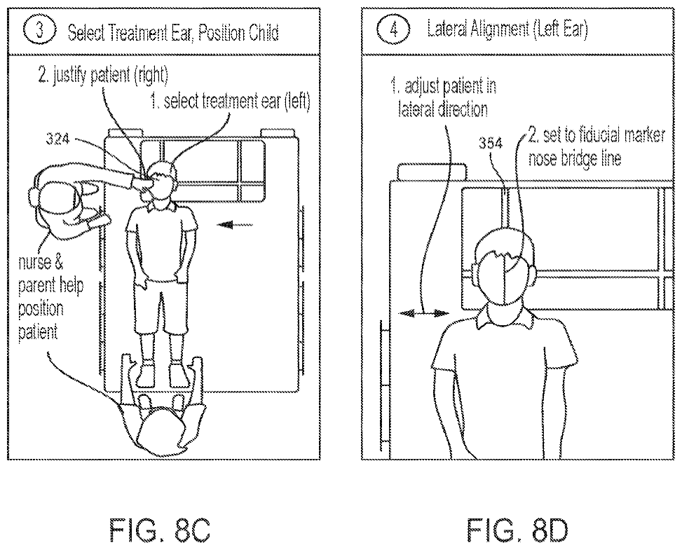

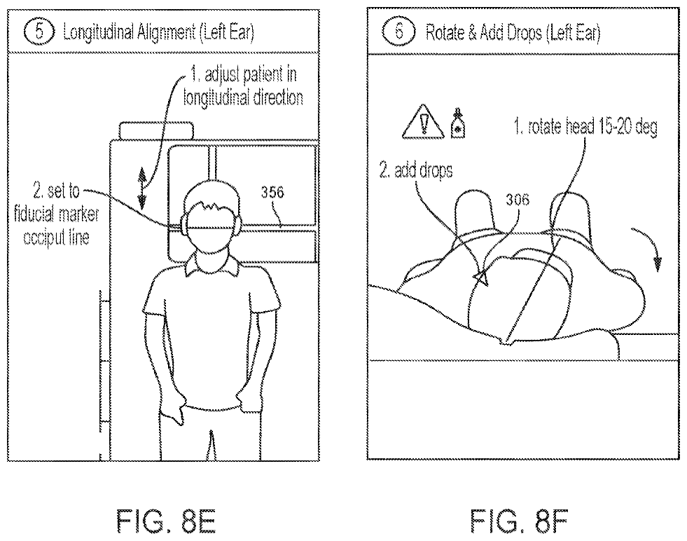

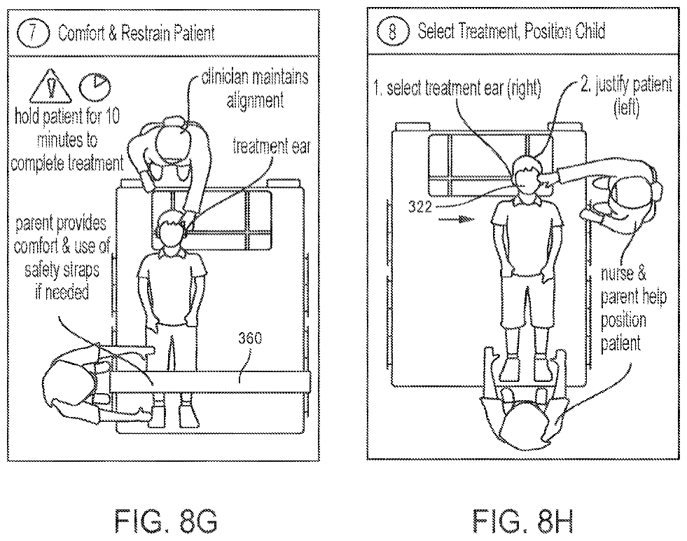

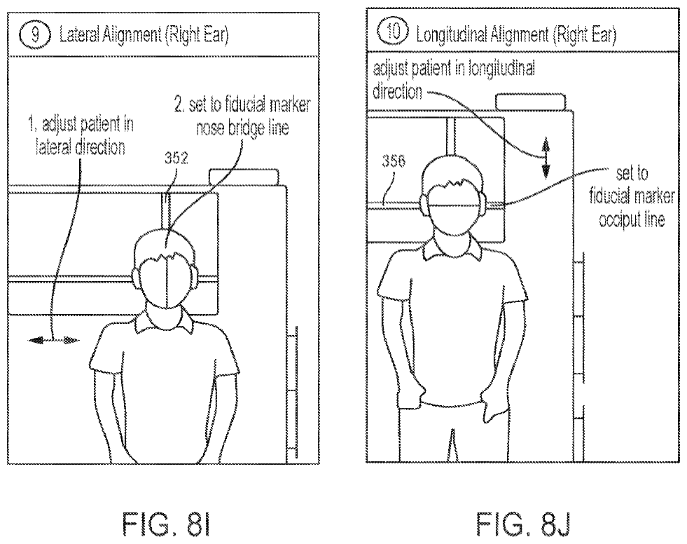

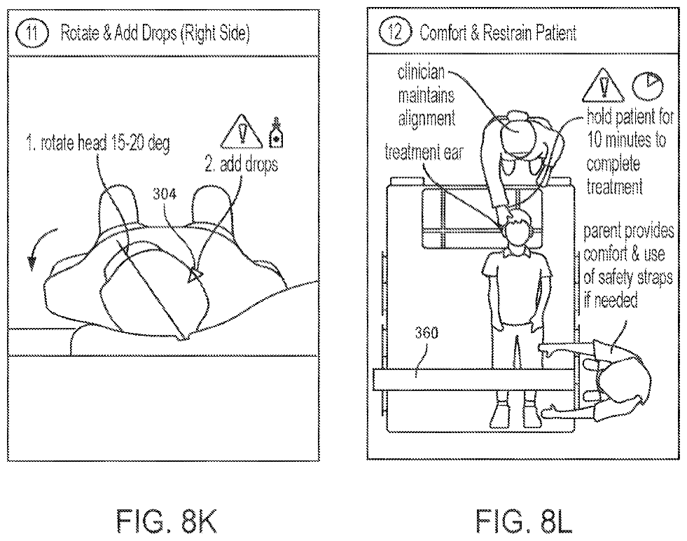

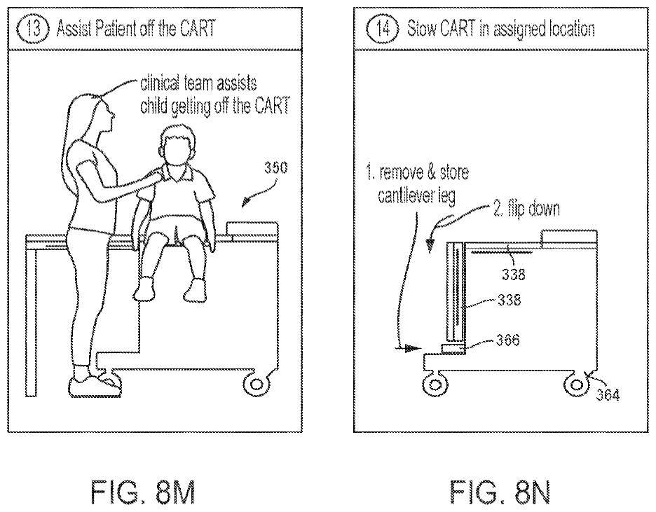

FIGS. 8A-8N illustrates a treatment procedure for administering a magnetic therapeutic agent to a patient through the ear canal on a medical cart 350. FIG. 8A shows the medical cart 350 secured in place by flipping up the hinged upper bed platform 338, deploying the cantilever leg 366, and locking the caster brakes 364. FIG. 8B shows a clinician placing a patient on the medical cart with the patient's head on the headrest 326. FIG. 8C shows the clinician aligning the patient's head in the left ear administration position 324 FIG. 8D shows the patient's head aligned according to the vertical second position indicator 354, where the bridge of the patient's nose is vertically aligned with the vertical second position indicator 354. FIG. 8E shows the patient's head aligned according to the horizontal/transverse position indicator 356, where the eyes of the patient are aligned with horizontal/transverse position indicator 356. FIG. 8F shows the patient's head rotated approximately 15 to 20 degrees positioning the left ear 306 above the horizontal plane. After the patient's head is rotated into the administration position, the clinician administers the magnetic therapeutic agent to the patient. FIG. 8G shows the clinician securing the patient with the security harness 360 and ensuring that the patient remains in the administration position for the predetermined treatment period. FIG. 8H shows the clinician carefully transferring the patient to the right ear administration position 322. FIG. 8I shows the patient's head aligned according to the vertical first position indicator 352 (right car), where the bridge of the patient's nose is vertically aligned with the vertical first position indicator 352. FIG. 8J shows the patient's head aligned according to the horizontal/transverse position indicator 356, where the eyes of the patient are aligned with horizontal/transverse position indicator 356. FIG. 8K shows the patient's head rotated approximately 15 to 20 degrees positioning the right ear 304 above the horizontal plane. After the patient's head is rotated into the administration position, the clinician administers the magnetic therapeutic agent to the patient. FIG. 8L shows the clinician securing the patient with the security harness 360 and ensuring that the patient remains in the administration position for the predetermined treatment period. FIG. 8M shows the clinician aiding the patient off of the medical cart. FIG. 8N shows a storage position of the medical cart where the hinged upper bed platform 338 is folded into a storage position and the cantilever leg 366 is retracted. The caster brakes 364 may be released and the cart may be placed in the storage location.

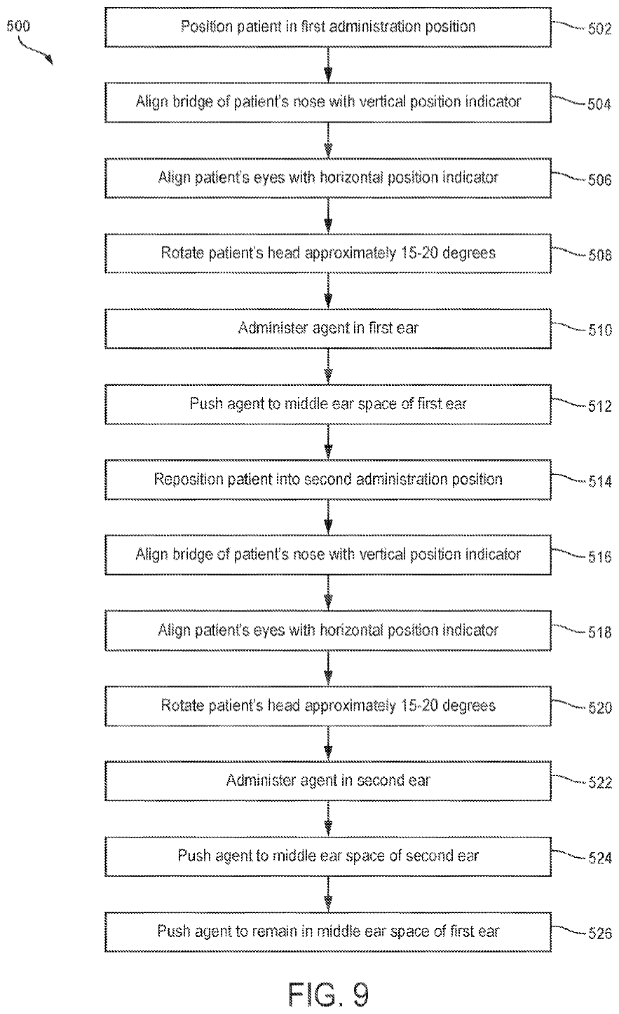

FIG. 9 shows a flow diagram for administering a magnetic therapeutic agent to a patient on a medical cart. The patient is positioned 502 on the medical cart in a first administration position. The patient's head is positioned in the first head contour of the headrest. The bridge of the patient's nose is aligned 504 with the first position vertical indicator of the headrest. The patient's eyes are aligned 506 with the horizontal indicator of the headrest. The patient's head is rotated 508 approximately 15-20 degrees, so that the first administration ear is above the horizontal plane. The magnetic therapeutic agent is administered 510, by the clinician, into the first administration ear. The therapeutic agent is provided a predetermined waiting period while it is pushed 512, by a magnetic force produced by the magnetic assembly, into the middle ear space of the first administration ear. After the predetermined waiting period, the patient is transferred or repositioned 514 into the second administration position for the second administration ear. The bridge of the patient's nose is aligned 516 with the second position vertical indicator of the headrest. The patient's eyes are re-aligned 518 with the horizontal indicator of the headrest. The patient's head is rotated 520 approximately 15-20 degrees so that the second administration ear is above the horizontal plane. The magnetic therapeutic agent is administered 522, by the clinician, into the second administration ear. The therapeutic agent is provided a predetermined waiting period while it is pushed 524, by a magnetic force produced by the magnetic assembly, into the middle ear space of the second administration ear. Throughout the duration that the patient is in the second administration position, the therapeutic agent administered to the first car is pushed, by magnetic force produced by the magnetic assembly, and maintained 526 in the target middle ear space of the first car.

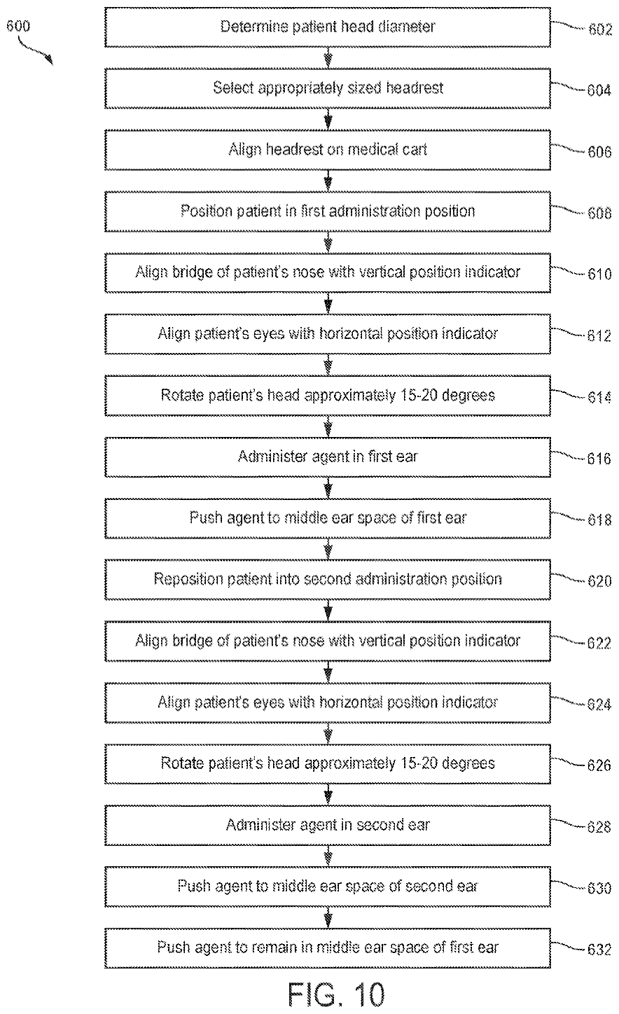

FIG. 10 shows a flow diagram for configuring a medical cast and administering a magnetic therapeutic agent to a patient on the medical cart. The diameter of the patient's bead is measured 602 by the clinician. The patient's head diameter is used to determine and select 604 an appropriately sized headrest, such that the patient's head conforms snuggly to the head contour positions of the headrest. The headrest is aligned 606 according to position markers on the headrest with complementary markers on the medical cart. The position markers ensure that the alignment grid of the headrest correctly indicates the desired patient alignment positions. The patient is positioned 608 on the medical cart in a first administration position. The patient's head is positioned in the first head contour of the headrest. The bridge of the patient's nose is aligned 610 with the first position vertical indicator of the headrest The patient's eyes are aligned 612 with the horizontal indicator of the headrest The patient's head is rotated 614 approximately 15-20 degrees, so that the first administration ear is above the horizontal plane. The magnetic therapeutic agent is administered 616, by the clinician, into the first administration ear. The therapeutic agent is provided a predetermined waiting period while it is pushed 618, by a magnetic force produced by the magnetic assembly, into the middle ear space of the first administration ear. After the predetermined waiting period, the patient is transferred or repositioned 620 into the second administration position for the second administration ear The bridge of the patient's nose is aligned 622 with the second position vertical indicator of the headrest. The patient's eyes are re-aligned 624 with the horizontal indicator of the headrest. The patient's head is rotated 626 approximately 15-20 degrees so that the second administration ear is above the horizontal plane. The magnetic therapeutic agent is administered 628, by the clinician, into the second administration ear. The therapeutic agent is provided a predetermined waiting period while it is pushed 630, by a magnetic force produced by the magnetic assembly, into the middle ear space of the second administration ear. Throughout the duration that the patient is in the second administration position, the therapeutic agent administered to the first ear is pushed, by magnetic force produced by the magnetic assembly, and maintained 632 in the target middle ear space of the first ear.

Various aspects of the subject matter described herein are set out in the following numbered examples.

Example 1: A magnetic agent delivery cart comprising: an upper bed platform, a magnetic assembly located under the upper bed platform, wherein the magnet assembly is centered underneath the longitudinal axis of the upper bed platform and aligned at a proximal end of the magnetic agent delivery cart; one or more side fairings configured to hide the magnetic assembly, wherein the one or more side fairings is removable; a headrest located on top of the upper bed platform and positioned above the center of the magnet assembly; the headrest further comprising: a first head contour and a second head contour, wherein the first head contour is configured for the patient in a first administration position and the second head contour is configured for the patient in a second administration position; and an alignment grid configured according to the first and second administration position and the location of the magnet assembly wherein the alignment grid comprises a first position vertical line that corresponds to the sagittal plane of the patient in the first administration position, a second position vertical line that corresponds to the sagittal plane of the patient in the second administration position, and a horizontal line that corresponds to the transverse plane along the patient's eyes.

Example 2: The magnetic agent delivery cart of Example 1, wherein the magnetic assembly comprises a magnet army of a plurality of magnet pillars, and wherein the plurality of pillars are positioned in a row and column grid structure with a predetermined number of rows and columns.

Example 3: The magnetic agent delivery cart of Example 2, wherein the predetermined number of rows and columns are determined based on a plurality of magnetic forces exerted on a magnetic therapeutic agent.

Example 4: The magnetic agent delivery cart of Example 3, wherein a first magnetic force is based on the right ear in the first administration position, a second magnetic force is based on the left ear in the first administration position, a third magnetic force is based on the right ear in the second administration position, and a fourth magnetic force is based on the left ear in the second administration position.

Example 5: The magnetic agent delivery cant of any of the Examples 2-4, wherein each of the plurality of pillars further comprise a plurality of magnetic discs, wherein the plurality of magnetic discs are bonded together and magnetized in the same direction.

Example 6: The magnetic agent delivery cart of Example 5, wherein each of the plurality of pillars comprise ne number of discs.

Example 7: The magnetic agent delivery cart of any of the Examples 1-6, further comprising: patient securing harness; one or more casters comprising a brake to lock the caster in place; and one or more cantilever legs configured to fold from a first storage position to a second position, wherein the second position anchors the magnetic agent delivery cart in place.

Example 8: The magnetic agent delivery cart of any of the Examples 1-7, wherein the headrest further comprises ne or more alignment marking that are configured to position or affix the headrest accord ng to the magnet assembly.

Example 9. The magnetic agent delivery cart of any of the Examples 1-8, wherein the headrest is removable and is selected from a plurality of headrests that contour to different head diameters.

Example 10: A system for delivering a magnetic agent to a patient through the ear canal, the system comprising: a magnetic therapeutic agent comprising a magnetic nanoparticles attached to a therapy: a medical cart comprising an upper bed platform; a magnetic assembly located under the upper bed platform, wherein the magnet assembly is centered underneath the longitudinal axis of the upper bed platform and aligned at a proximal end of the magnetic agent delivery cart; one or more side fairings configured to hide the magnetic assembly, wherein the one or more side fairings is removable; a headrest located on top of the upper bed platform and positioned above the center of the magnet assembly, the headrest further comprising: a first bead contour and a second head contour, wherein the first head contour is configured for the patient in a first administration position and the second head contour is configured for the patient in a second administration position; and an alignment grid configured according to the first and second administration position and the location of the magnet assembly, wherein the alignment grid comprises a first position vertical line that corresponds to the sagittal plane of the patient in the first administration position, a second position vertical line that corresponds to the sagittal plane of the patient in the second administration position, and a horizontal line that corresponds to the transverse plane along the patient's eyes.

Example 11: The system for delivering a magnetic agent to a patient through the ear canal of Example 10, wherein the magnetic assembly comprises a magnet array of a plurality of magnet pillars, and wherein the plurality of pillars are positioned in a row and column grid structure with a predetermined number of rows and columns.

Example 12: The system for delivering a magnetic agent to a patient through the ear canal of Example 11, wherein the predetermined number of rows and columns are determined based on a plurality of magnetic forces exerted on the magnetic therapeutic agent.

Example 13 The system for delivering a magnetic agent to a patient through the ear canal of Example 12, wherein a first magnetic force is based on the right ear in the first administration position, a second magnetic force is based on the left ear in the first administration position, a third magnetic force is based on the right ear in the second administration position, and a fourth magnetic force is based on the left ear in the second administration position.

Example 14: 14. The system for delivering a magnetic agent to a patient through the ear canal of any of the Examples 10-13, wherein each of the plurality of pillars further comprise a plurality of magnetic discs, wherein the plurality of magnetic discs are bonded together and magnetized in the same direction.

Example 15: The system for delivering a magnetic agent to a patient through the ear canal of Example 14, wherein each of the plurality of pillars comprises the same number of discs.

Example 16: The system for delivering a magnetic agent to a patient through the ear canal of any of the Examples 10-15, further comprising: patient securing harness; one or more casters comprising a brake to lock the caster in place; and one or more cantilever legs configured to fold from a first storage position to a second position, wherein the second position anchors the magnetic agent delivery cart in place.

Example 17. The system for delivering a magnetic agent to a patient through the ear canal of any of the Examples 10-16, wherein the headrest further comprises one or more alignment marking that are configured to position or affix the headrest according to the magnet assembly.

Example 18: The system for delivering a magnetic agent to a patient through the ear canal of any of the Examples 10-17, wherein the headrest is removable and is selected from a plurality of headrests that contour to different head diameters.

Example 19: A method for delivering a magnetic agent to a patient through the ear canal, the method comprising: positioning a patient on a medical cart in a first administration position, wherein the medical cart comprises a magnet assembly and a headrest, and wherein the headrest comprises a first head contour and a second head contour, and wherein the headrest comprises a horizontal alignment line, first position vertical alignment line, and a second position vertical alignment line; aligning the bridge of the patient's nose with the vertical first position indicator; aligning the patient's eyes in the horizontal plane with a horizontal position indicator rotating the patient's head so that a first administration ear is above the horizontal plane; administering a magnetic therapeutic agent to the first administration ear; pushing, by the magnet assembly, a magnetic therapeutic agent to a middle ear space of the patient's first ear; repositioning the patient on the medical cart in a second administration position; aligning the bridge of the patient's nose with the vertical second position indicator; aligning the patient's eyes in the horizontal plane with a horizontal position indicator; rotating the patient's head so that a second administration ear is above the horizontal plane; administering the magnetic therapeutic agent to the second administration ear; pushing, by the magnetic assmebly, the magnetic therapeutic agent to a middle ear space of the patient's second ear; pushing, by magnet assembly, the magnetic therapeutic agent in the first administration ear so that is remains in the middle ear space of the patient's second ear.

Example 20: A method for delivering a magnetic agent to a patient through the ear canal, the method comprising determining, based on the diameter of a patient's head, a headrest that conforms the patient's head, wherein the headrest comprises a first head contour and a second head contour, and wherein the headrest comprises a horizontal alignment line, first position vertical alignment line, and a second position vertical alignment line; aligning the headrest on the medical cart based on one or more alignment marker that is relative to the magnet assembly, positioning the patient on the medical cart in a first administration position, wherein the medical cart comprises a magnet assembly and the headrest; aligning the bridge of the patient's nose with the vertical first position indicator; aligning the patient's eyes in the horizontal plane with a horizontal position indicator; rotating the patient's head so that a first administration ear is above the horizontal plane; administering a magnetic therapeutic agent to the first administration ear; pushing, by the magnet assembly, a magnetic therapeutic agent to a middle ear space of the patient's first ear; repositioning the patient on the medical cart in a second administration position; aligning the bridge of the patient's nose with the vertical second position indicator; aligning the patient's eyes in the horizontal plane with a horizontal position indicator; rotating the patient's head so that a second administration ear is above the horizontal plane; administering the magnetic therapeutic gent to the second administration car; pushing, by the magnet assembly, the magnetic therapeutic middle ear space of the patient's second ear; and pushing, by the magnet assembly, the magnetic therapeutic agent in the first administration ear so that is remains in the middle ear space of the patient's second ear.

Although specific aspects have been disclosed in detail above, the description as merely for illustrative purposes It should be appreciated, therefore, that many aspects of the invention were described above by way of example only and are not intended as required or essential elements of the invention unless explicitly stated otherwise. Modifications of, and equivalent steps corresponding to, the disclosed aspects of the exemplary aspects, in addition to those described above, can be made by a person of ordinary skill in the art, having the benefit of this disclosure, without departing from the spirit and scope of the invention defined in the following claims, the scope of which is to be accorded the broadest interpretation so as to encompass such modifications and equivalent structures.

In the present disclosure, like reference characters designate like or corresponding parte throughout the several views of the drawings.

All patents, patent applications, publications, or offer disclosure material mentioned herein, are hereby incorporated by reference in their entirety as if each individual reference was expressly incorporated by reference respectively. All references, and any material, or portion thereof, that are said to be incorporated by reference herein are incorporated herein only to the extent that the incorporated material does not conflict with existing definitions, statements, or other disclosure material set forth in this disclosure. As such, and to the extent necessary, the disclosure as set forth herein supersedes any conflicting material incorporated herein by reference and the disclosure expressly set forth in the present application controls.

The present disclosure has been described with reference to various examples and illustrative aspects. The aspects described herein are understood as providing illustrative features of varying detail of various aspects of the disclosed invention; and therefore, unless otherwise specified, it is to be understood that, to the extent possible, one or more features, elements, components, constituents, ingredients, structures, modules, and/or aspects of the disclosed aspects may be combined, separated, interchanged, and/or rearranged with or relative to one or more other features, elements, components, constituents, ingredients, structures, modules, and/or aspects of the disclosed aspects without departing from the scope of the disclosed invention. Accordingly, it will be recognized by persons having ordinary skill in the art that various substitutions, modifications or combinations of any of the example aspects may be made without departing from the scope of the invention. In addition, persons skilled in the art will recognize, or be able to ascertain using no more than routine experimentation, many equivalents to the various aspects of the invention described herein upon review of the present disclosure. Thus, the present disclosure is not limited by the description of the various aspects, but rather by the claims.

Those skilled in the art will recognize that, in general, terms used herein, and especially in the appended claims (e.g., bodies of the appended claims) are generally intended as “open” terms (e.g, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g, “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having shill in the art would understand the convention (e.g., “a system having at least one of A, B, or C” would include but not be limited to systems that have A alone, B alone, C alone, A and B together, A and C together, B and (together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless contest dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although claim recitations are presented in a sequence(s), it should be understood that the various operations may be performed in other orders than those which are described, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past tense adjectives are generally not intended to exclude such variants, unless context dictates otherwise.

It is worthy to note that any reference to “one aspect,” “on aspect,” “an exemplification,” “one exemplification,” and the like means that a particular feature, structure, or characteristic described in connection with the aspect is included in at least one aspect. Thus, appearances of the phrases “in one aspect” “in an aspect,” “in an exemplification,” and “in one exemplification” in various places throughout the present disclosure are not necessarily all referring to the same aspect. Furthermore, the particular features, structures or characteristics may be combined in any suitable manner in one or more aspects.

As used herein, the singular form of “a”, “an”, and “the” include the plural references unless the context clearly dictates otherwise.

Directional phrases used herein, such as, for example and without limitation, top, bottom, left, right, lower, upper, front, back, and variations thereof, shall relate to the orientation of the elements shown in the accompanying drawing and are not limiting upon the claims unless otherwise stated.

The terms “about” “approximately” as used in the present disclosure, unless otherwise specified, means an acceptable error for a particular value as determined by one of ordinary skill in the art, which depends in part on how the value is measured or determined. In certain aspects, the term “about” or “approximately” means within 1, 2, 3, or 4 standard deviations. In certain aspects, the term “about” or “approximately” means within 50%, 200%, 105%, 100%, 9%, 8%, 6%, 5%, 4%, 3%, 2%, 1%, 0.5%, 0.05%% of a given value of range.

In the present disclosure, unless otherwise indicated, all numerical parameters are to be understood as being prefaced and modified in all instances by the term “about,” in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described herein should at least be construed in light of the number of reported significant digits and by applying ordinary rounding techniques.

Any numerical range recited herein includes all sub-ranges subsumed within the recited range. For example, a range of “1 to 100” includes all so ranges between (and including) the recited minimum value of 1 and the recited maximum value of 100, that is, having a minimum value equal to or greater than 1 and a maximum value equal to or less than 100. Also, all ranges recited herein are inclusive of the end points of the recited ranges. For example, a range of “1 to 100” includes the end points 1 and 100. Any maximum numerical limitation recited in the present disclosure is intended to include all lower numerical limitations subsumed therein, and any minimum numerical limitation recited in the present disclosure is intended to include all higher numerical limitations subsumed therein. Accordingly, Applicant reserves the right to amend the present disclosure, including the claims, to expressly recite any sub-range subsumed within the ranges expressly recited. All such ranges are inherently described in the present disclosure.

Any patent application, patent, non-patent publication, or other disclosure material referred to in the present disclosure and/or listed in any Application Data Sheet is incorporated by reference herein, to the extent that the incorporated materials is not inconstant herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs As a result, a system that “comprises,” “has,” “includes” or “contains” one or more elements possesses those one of more elements, but is not limited to possessing only those one or more elements. Likewise, an element of a system, device, or apparatus that “comprises,” “has,” “includes” or “contains” one or more features possesses those one or more features, but is not limited to possessing only those one or more features.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

1. A magnetic agent delivery cart comprising:
an upper bed platform;
a magnetic assembly located under the upper bed platform, wherein the magnetic assembly is centered underneath the longitudinal axis of the upper bed platform and aligned at a proximal end of the magnetic agent delivery cart;
one or more side fairings configured to hide the magnetic assembly, wherein the one or more side fairings is removable;
a headrest located on top of the upper bed platform and positioned above the center of the magnetic assembly, the headrest further comprising:
a first head contour and a second head contour, wherein the first head contour is configured for the patient in a first administration position and the second head contour is configured for the patient in a second administration position:
and an alignment grid configured according to the first and second administration position and the location of the magnet assembly, wherein the alignment grid comprises a first position vertical line that corresponds to the sagittal plane of the patient in the first administration position, a second position vertical line that corresponds to the sagittal plane of the patient in the second administration position, and a horizontal line that corresponds to the transverse plane along the eyes of the patient.

2. The magnetic agent delivery cart of claim 1, wherein the magnetic assembly comprises a magnet array of a plurality of magnet pillars, and wherein the plurality of magnet pillars are positioned in a row and column grid structure with a predetermined number of rows and columns.

3. The magnetic agent delivery cart of claim 2, wherein the predetermined number of rows and columns are determined based on a plurality of magnetic forces exerted on a magnetic therapeutic agent.

4. The magnetic agent delivery cart of claim 3, wherein a first magnetic force is based on the right ear in the first administration position, a second magnetic force is based on the left ear in the first administration position, a third magnetic force is based on the right ear in the second administration position, and a fourth magnetic force is based on the left ear in the second administration position.

5. The magnetic agent delivery cart of claim 2, wherein each of the plurality of magnet pillars further comprise a plurality of magnetic discs, wherein the plurality of magnetic discs are bonded together and magnetized in the same direction.

6. The magnetic agent delivery cart of claim 5, wherein each of the plurality of pillars comprises the same number of discs.

7. The magnetic agent delivery cart of claim 1, further comprising:
a patient securing harness; one or more casters comprising a brake to lock the caster in place; and
one or more cantilever legs configured to fold from a first storage position to a second position, wherein the second position anchors the magnetic agent delivery cart in place.

8. The magnetic agent delivery cart of claim 1, wherein the headrest further comprises one or more alignment marking that are configured to position or affix the headrest according to the magnet assembly.

9. The magnetic agent delivery cart of claim 1, wherein the headrest is removable and is selected from a plurality of headrests that contour to different head diameters.

10. A system for delivering a magnetic agent to a patient through the ear canal, the system comprising:
a magnetic therapeutic agent comprising magnetic nanoparticles attached to a therapy;
a medical cart comprising:
an upper bed platform;
a magnetic assembly located under the upper bed platform, wherein the magnetic assembly is centered underneath the longitudinal axis of the upper bed platform and aligned at a proximal end of the medical cart:
one or more side fairings configured to hide the magnetic assembly, wherein the one or more side fairings is removable;
a headrest located on top of the upper bed platform and positioned above the center of the magnetic assembly, the headrest further comprising:
a first head contour and a second head contour, wherein the first head contour is configured for the patient in a first administration position and the second head contour is configured for the patient in a second administration position; and
an alignment grid configured according to the first and second administration position and the location of the magnetic assembly, wherein the alignment grid comprises a first position vertical line that corresponds to the sagittal plane of the patient in the first administration position, a second position vertical line that corresponds to the sagittal plane of the patient in the second administration position, and a horizontal line that corresponds to the transverse plane along the eyes of the patient.

11. The system for delivering a magnetic agent to a patient through the ear canal of claim 10, wherein the magnetic assembly comprises a magnet array of a plurality of magnet pillars, and wherein the plurality of pillars are positioned in a row and column grid structure with a predetermined number of rows and columns.

12. The system for delivering a magnetic agent to a patient through the ear canal of claim 11, wherein the predetermined number of rows and columns are determined based on a plurality of magnetic forces exerted on the magnetic therapeutic agent.

13. The system for delivering a magnetic agent to a patient through the ear canal of claim 12, wherein a first magnetic force is based on the right ear in the first administration position, a second magnetic force is based on the left ear in the first administration position, a third magnetic force is based on the right ear in the second administration position, and a fourth magnetic force is based on the left ear in the second administration position.

14. The system for delivering a magnetic agent to a patient through the ear canal of claim 11, wherein each of the plurality of pillars further comprise a plurality of magnetic discs, wherein the plurality of magnetic discs are bonded together and magnetized in the same direction.

15. The system for delivering a magnetic agent to a patient through the ear canal of claim 14, wherein each of the plurality of pillars comprises the same number of discs.

16. The system for delivering a magnetic agent to a patient through the ear canal of claim 10, further comprising:
a patient securing harness; one or more casters comprising a brake to lock the caster in place; and one or more cantilever legs configured to fold from a first storage position to a second position, wherein the second position anchors the magnetic agent delivery cart in place.

17. The system for delivering a magnetic agent to a patient through the ear canal of claim 10, wherein the headrest further comprises one or more alignment marking that are configured to position or affix the headrest according to the magnet assembly.

18. The system for delivering a magnetic agent to a patient through the ear canal of claim 10, wherein the headrest is removable and is selected from a plurality of headrests that contour to different head diameters.

19. (canceled)

20. (canceled)
